(19) United States
(12) Patent Application Publication
(10) Pub. No.: US 2011/0184327 A1
(43) Pub. Date: Jul. 28, 2011

(54) TRACHEOSTOMY DRESSING

(75) Inventors: Malcolm Cameron, Cambridgeshire (GB); Luke Cascarini, Beckenham (GB)

(73) Assignee: Cambridge University Hositals NHS Foundation Trust

(21) Appl. No.: 13/056,722

(22) PCT Filed: Jul. 31, 2009
(86) PCT No.: PCT/GB2009/050950

§ 371 Date: Apr. 12, 2011

(30) Foreign Application Priority Data
Aug. 1, 2008 (GB) 0814102.0

(51) Int. Cl. A61F 13/00; A61L 15/44

(52) U.S. Cl. 602/43; 602/48

(57) ABSTRACT

A tracheostomy dressing (1) which comprises an integral wound locating device (4) for locating and applying pressure to a healing tracheostomy wound.

This invention relates to a tracheostomy dressing which enables patients to easily locate a healing tracheostomy wound on the neck following decanulation (removal of a tracheostomy tube).

Temporary tracheostomies are used in many areas of surgery and anaesthesia. They are removed (that is, the patient is decanulated) once their usefulness has passed. Following decanulation, it is desirable for the tracheostomy wound to heal as quickly as possible as delay in healing can be problematic for a patient. Delay may be caused by persistent passage of expired air via the tracheostomy wound rather than the larynx (voicebox). If this occurs, the healing wound is disrupted, literally blown open, necessarily slowing down the healing process.

Traditionally, patients have been instructed to apply firm pressure with finger tips or knuckles over the tracheostomy wound to prevent the unwanted passage of air via the healing wound. Even with careful guidance, many patients find it very difficult to locate the wound easily, in particular because the patient cannot see the wound, thus reducing the effectiveness of this action. Dressings are placed over the healing wound which preclude exact location of the wound below by the patient. Such dressings may be bulky or the patients' manual dexterity or sensation may be impaired. In addition, the dressings often cause excoriation of the skin. Consequently, although the patient may attempt to comply with instructions, if pressure is not applied in the correct position the action is futile. If pressure is incorrectly applied by the patient, the dressing can be dislodged with the resultant passage of air and results in exudates passing through the wound. This situation is clearly disadvantageous and significantly impairs the healing process and increases the risk of infection.

GB 2413767 (Queen Victoria Hospital NHS Foundation Trust) discloses a toggle device for use in combination with existing dressings enabling a patient to locate and apply pressure to a healing tracheostomy wound.

According to one aspect of the invention, there is provided a tracheostomy dressing which comprises an integral wound locating device for locating and applying pressure to a healing tracheostomy wound.

The invention provides a dressing which can be simply applied to a patient without the need to combine a separate wound locating device with wound dressings. Such an integrated dressing provides a significant number of advantages primarily relating to patient comfort and optimised healing. For example, the integrated dressing effectively prevents air and exudate from escaping through the tracheostomy wound during high-pressure expiration such as talking, coughing or sneezing (i.e. the integrated device provides a more effective seal or vacuum when pressure is applied). This will result in greater patient comfort because current tracheostomy patients experience pain and discomfort when air/exudate passes through the wound site. The integrated device provides a product which is less likely to cause ingress of pathogens and therefore reduces the likelihood of infection. The integrated device also results in faster speech restoration because air is correctly directed through the larynx/voicebox rather than through the wound site. Furthermore, the dressing of the invention is more easily and quickly applied to a patient by nursing staff which not only enhances the healing process but also reduces the amount of valuable time used by the nursing staff in this procedure.

References herein to “wound locating device” shall be interpreted as meaning a device for locating and applying pressure to a healing tracheostomy wound.

References herein to “dressing” include all medical products which are intended to be applied to a wound and aid in the healing of said wound and prevent further harm or damage. Typically, the dressing should be sterile, breathable, and encourage a moist healing environment.

Examples of suitable dressings include bandages and plasters. Particular dressings which may be suitable for use with the integral wound locating device are described in www.dressings.org and include Acticoat, Actisorb Silver 220, Algisite M, Allevyn, Aquacel, Bactigras, Biatain, Bioclusive, Biofilm, Blenderm, Blue line webbing, Bordered Granuflex, Calaband, Carbonet, Cavi-care, Cellacast Xtra, Cellamin, Cellona, Chlorhexitulle, Cica-Care, Cliniflex, Clinisorb, Coban, Coltapaste, Comfeel, Contreet Non Adhesive, Crevic, Cutinova Hydro, Debrisan, Delta-Cast, Delta-Lite S, Duoderm extra thin, Duoderm signal, Durapore, Elastocrepe, Elset, Flamazine, Fucidin Intertulle, Geliperm, Granuflex (Improved formulation), Granuflex extra thin, Granugel, Gypsona, Hypafix, Icthaband, Icthopaste, Inadine, Intrasite Gel, Iodoflex, Iodosorb, Jelonet, K-Band, K-Lite, K-Plus, Kaltocarb, Kaltostat, LarvE, Lestreflex, Lyofoam, Mefix, Melolin, Mepiform, Mepilex, Mepitac, Mepitel, Mepore, Mesorb, Mesitran, Metrotop, Microfoam, Micropore, Opsite Flexigrid, Opsite IV 3000, Orthoflex, Oxyzyme, Paratulle, Polymem, Profore, ProGuide, Promogran, Quinaband, Release, Scotchcast Plus, Scotchcast Softcast, Serotulle, Setopress, Silastic foam, Silicone N-A, Sofra-Tulle, Sorbsan, Spenco 2nd Skin, Spyroflex, Spyrosorb, Tarband, Tegaderm, Tegagel, Tegapore, Tegasorb, Telfa, Tensopress, Tielle, Transpore, Unitulle, Veinoplast, Veinopress, Versiva, Vigilon, Viscopaste PB7, Xelma and Zincaband.

In one embodiment, the dressing comprises a gauze, a film, a gel, a foam, a hydrocolloid, an alginate, a hydrogel or a polysaccharide paste, granule or bead. In a further embodiment, the dressing comprises a hydrocolloid material. In a yet further embodiment, the dressing comprises a Granuflex dressing (e.g. Bordered Granuflex, Granuflex (Improved formulation) or G ranuflex extra thin) or Duoderm dressing (e.g. Duoderm signal or Duoderm extra thin). Such dressings provide the advantage of adhering to the patient's skin but without causing abrasion or skin excoriation upon removal of the dressing. Additionally, the provision of a hydrocolloid based dressing provides an advantageous vacuum over the tracheostomy wound. Such a vacuum prevents the flow of air and/or exudate through the wound during high-pressure expiration such as talking, coughing or sneezing.

In one embodiment, the dressing may be impregnated with an agent designed to enhance sterility and/or healing. In a further embodiment, the dressing may be impregnated with one or more antiseptic chemicals.

It will be appreciated that references to “integral” include the sale of the dressing having the wound locating device attached rather than the wound locating device constituting a separate item of commerce. Therefore, in one embodiment, the dressing is constructed from a single piece together with the integral wound locating device. In an alternative embodiment, the wound locating device may be constructed separately from the dressing and bonded (e.g. via adhesive or otherwise) to the tracheostomy dressing. In such an embodiment, the wound locating device may be constructed by many methods such as injection moulding or lathe cutting. In such an embodiment the wound locating device may be constructed from a single piece. Constructing the wound locating device from a single piece is advantageous because it simplifies the manufacturing process, reduces cost and may make the wound locating device more hygienic.

In one embodiment, the integral wound locating device is as described in GB 2413767. For example, the wound locating device consists of a shaped contact plate (e.g. having a substantially square outline and a curved cross section) onto which a short stalk is attached anteriorly, wherein the top of the stalk comprises a finger button.

In an alternative embodiment, the integral wound locating device is substantially square, rectangular or circular in shape. In a yet further embodiment, the integral wound locating device is substantially circular in shape. In a further embodiment, the wound locating device is approximately 0.5 to 4 cm in diameter. It will be appreciated that smaller sized devices would be envisaged for children (e.g. approximately 0.5 to 1 cm in diameter).

In one embodiment, the integral wound locating device additionally comprises a textured portion. It will be appreciated that the textured portion may comprise any feature which would have a surface feel which is distinct from the surrounding area. Examples of textured portions include but are not limited to a recessed or concave portion, a ribbed portion, a bobbled portion, a studded portion or a portion constructed from non-slip material.

This embodiment, while being contrary to the protruding toggle described in GB 2413767, provides a number of advantages. For example, the textured portion of the wound locating device can be simply found by moving the tip of a finger over the dressing. Furthermore, the textured portion is more subtle than the toggle described in GB 2413767 which enhances patient compliance and acceptance. The toggle embodiment could easily be knocked and cause mis-alignment of the dressing with respect to the tracheostomy wound. By contrast, the dressing of the invention is less likely to be dislodged because it is substantially flat. Additionally, the textured portion provides a configuration which permits simple moulding from a single piece and is therefore suitable for integral construction within a dressing.

In a further embodiment, the textured portion is a recessed or concave portion. Such an embodiment is advantageous because it makes the wound locating device easier to find due to the shape of the textured portion, and, once found, the tip of a patient's finger is less likely to slip off the wound locating device because the finger tip is located by the recessed or concave shape of the textured portion. Furthermore, a recessed or concave portion is even less likely to snag on for instance clothes and jewellery than the toggle described in GB 2413767 because it does not protrude significantly from the dressing and does not provide an upstanding feature to catch clothing or the like.

In a further embodiment, the textured portion is a ribbed portion. Such an embodiment is also advantageous because it makes the wound locating device easier to find due to the shape of the textured portion, and, once found, the tip of a patient's finger is less likely to slip off the wound locating device because of the resistance created by the ribbed portion. Furthermore, a ribbed portion is less likely to snag on for instance clothes and jewellery than the toggle described in GB 2413767 because it does not protrude from the dressing to the extent that the toggle does.

In a further embodiment, the textured portion is both recessed or concave and ribbed.

It will be appreciated that the textured portion may be any geometric shape suitable to allow rapid sensory knowledge of the exact location of the wound locating device. In a further embodiment, the textured portion is substantially square, rectangular or circular in shape. In a yet further embodiment, the textured portion is substantially circular in shape.

According to a second aspect of the invention, there is provided a tracheostomy wound locating device for locating and applying pressure to a healing tracheostomy wound, wherein said wound locating device comprises a textured portion.

It will be appreciated that the features described hereinbefore for the textured portion apply equally to this aspect of the invention.

The wound locating device may be constructed from many materials. It should be firm but not necessarily rigid, indeed a slightly flexible material may be preferable for patient comfort and improved function. A slightly flexible material would conform better to the patients' surface anatomy and reduce the risk of air escaping from the edges of the wound locating device. In particular, it has been observed that many patients often present with severe malnourishment which results in fairly deep recesses within their neck regions. In one embodiment, the wound locating device is constructed from a flexible material. In a further embodiment, the wound locating device is constructed from a polyurethane, polyethylene, thermoplastic elastomeric (TPE) material, thermoplastic rubber (TPR) material, a silicone material or a styrene ethylene butylene styrene (SEBS) material.

In one embodiment, the wound locating device is transparent. This embodiment provides the advantage of being able to accurately position the dressing over the tracheostomy wound.

According to a further aspect of the invention, there is provided a method of applying a tracheostomy dressing as defined hereinbefore wherein said method comprises the steps of:
(a) application of said dressing to a tracheostomy wound; and
(b) application, to said dressing, of a wound locating device as defined hereinbefore via an adhesive layer.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

The invention will now be described, by way of example only, with reference to the accompanying drawings, in which:

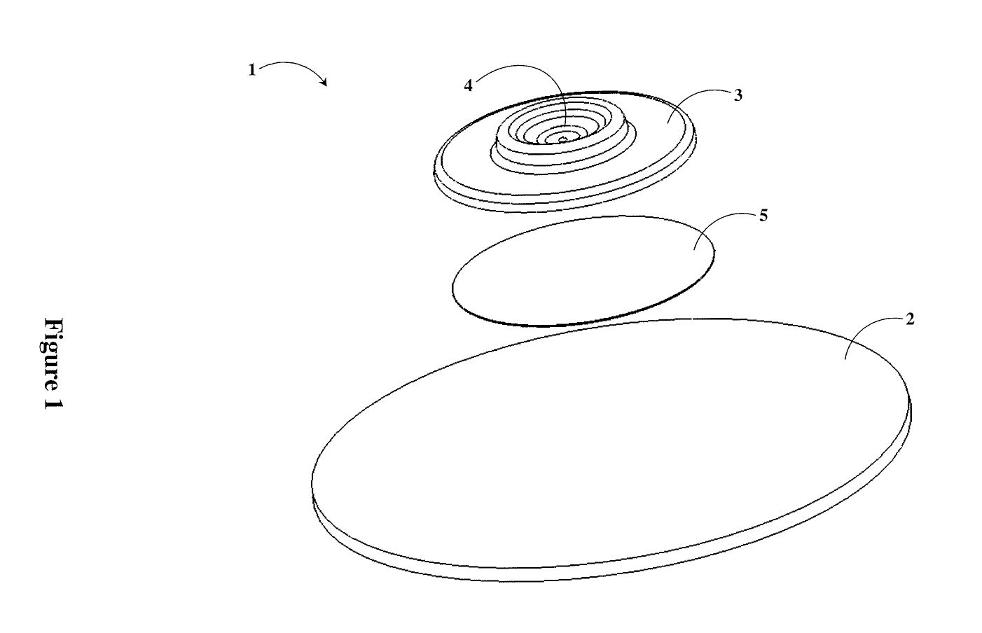

FIG. 1 shows an exploded view of the components of the tracheostomy dressing according to one embodiment of the invention;

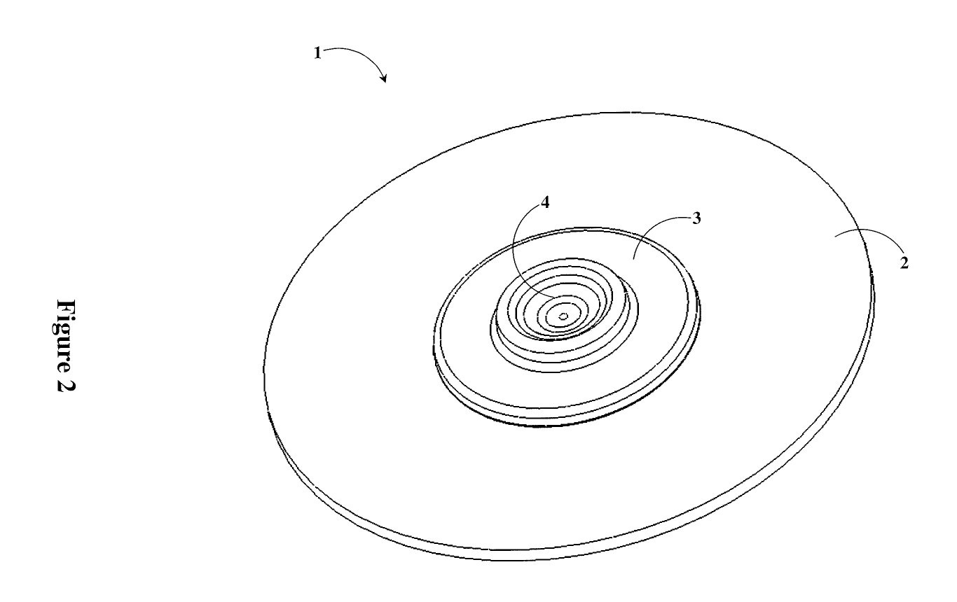

FIG. 2 shows a perspective view of the tracheostomy dressing according to one embodiment of the invention;

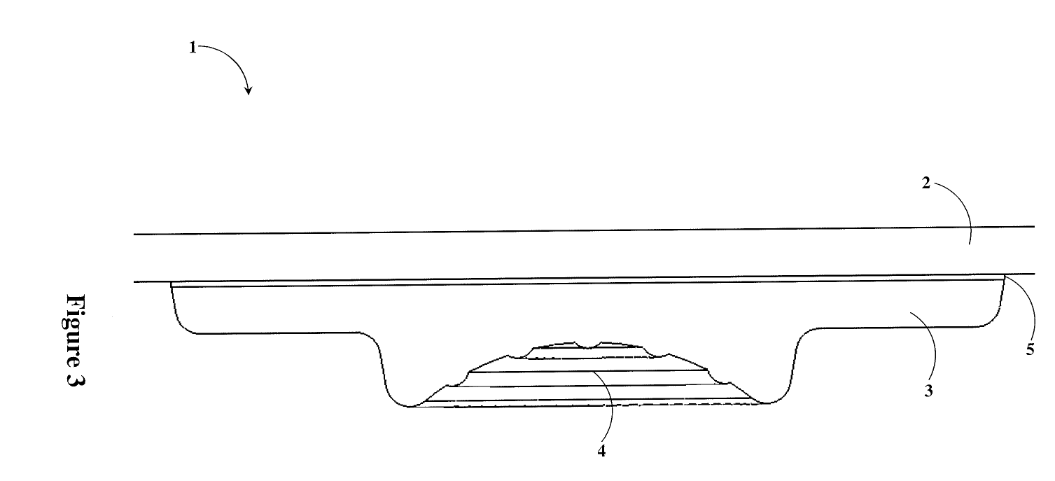

FIG. 3 shows a side view of the tracheostomy dressing according to one embodiment of the invention; and

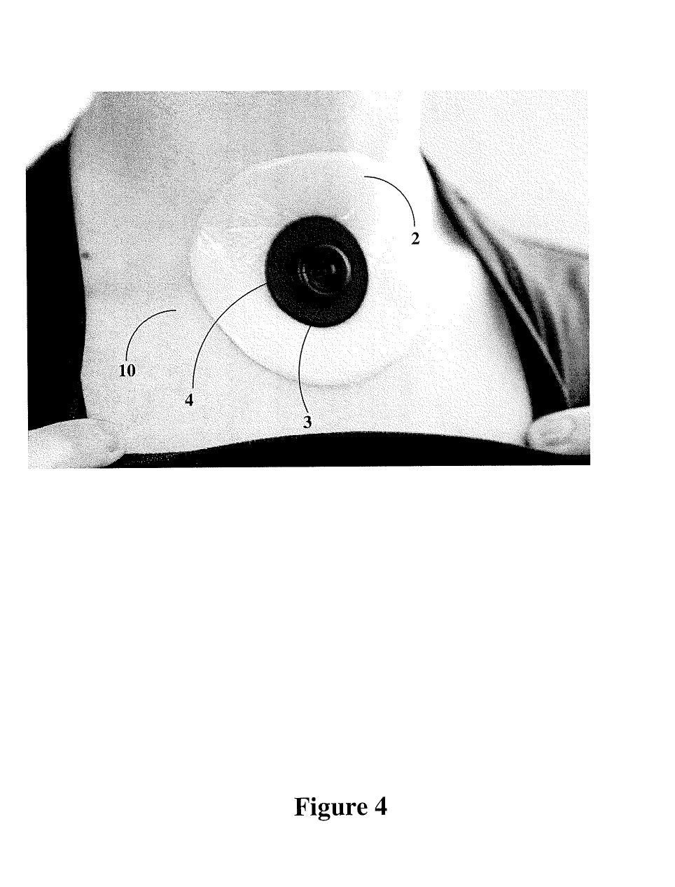

FIG. 4 shows a photograph demonstrating an in situ position of the tracheostomy dressing according to one embodiment of the invention.

Referring first to FIG. 1, a tracheostomy dressing shown generally as 1, comprises a dressing portion 2, a wound locating portion 3 and an adhesive layer 5 for attachment of the wound locating portion 3 to the dressing portion 2. In the embodiment shown in FIG. 1, the wound locating portion 3 comprises a series of circular ribs which create a recessed or concave portion 4.

FIG. 2 shows a perspective view of the embodiment shown in FIG. 1, FIG. 3 shows the advantageous flattened profile of the device as hereinbefore discussed and FIG. 4 shows a photographic image of the embodiment shown in FIG. 1 in situ on a model patient 10.

In an alternative embodiment to that described in FIGS. 1 to 4 an integral tracheostomy dressing is provided wherein the adhesive layer 5 is omitted and the wound locating portion 3 is moulded integrally with the dressing portion 2.

In use, the tracheostomy dressing is applied to the wound of a patient following decanulation. During use, the patient readily feels the recessed portion 4 which is easily located in an integral position within the wound dressing. This enables firm pressure to be applied in the correct position (that is, directly over the wound) during times when air could preferentially pass via the healing wound rather than the larynx. This occurs during high-pressure expiration such as talking, coughing or sneezing. The pressure on the recessed portion 4 and the ribs means that the finger tip of the patient is less likely to slip off the wound locating portion 3 once it is located.

It is expected that the tracheostomy dressing will be replaced everyday for 1-2 days following decanulation and every 2-3 days thereafter. Thus, it is expected that each patient will typically require approximately 10 dressings.

1. A tracheostomy dressing which comprises an integral wound locating device for locating and applying pressure to a healing tracheostomy wound, wherein the integral wound locating device comprises a textured portion.

2. A tracheostomy dressing as defined in claim 1, wherein the tracheostomy dressing comprises a hydrocolloid material.

3. (canceled)

4. A tracheostomy dressing as defined in claim 1, wherein the tracheostomy dressing is impregnated with one or more antiseptic chemicals.

5-7. (canceled)

8. A tracheostomy dressing as defined in claim 26 wherein the textured portion is a recessed or concave portion, a ribbed portion, or a portion constructed from non-slip material.

9. A tracheostomy dressing as defined in claim 8 wherein the textured portion is a recessed or concave portion.

10. A tracheostomy dressing as defined in claim 8 wherein the textured portion is a ribbed portion.

11. A tracheostomy dressing as defined in claim 8 wherein the textured portion is both recessed or concave and ribbed.

12. A tracheostomy dressing as defined in claim 1 wherein the textured portion is square, rectangular or circular in shape.

13. A tracheostomy dressing as defined in claim 12 wherein the textured portion is circular in shape.

14. A tracheostomy dressing as defined in claim 1 wherein the wound locating device is constructed from a flexible material.

15. A tracheostomy dressing as defined in claim 14 wherein the wound locating device is constructed from a polyurethane, polyethylene, thermoplastic elastomeric (TPE) material, thermoplastic rubber (TPR) material, a silicone material or a styrene ethylene butylene styrene (SEBS) material.

16. A tracheostomy dressing as defined in claim 1 wherein the wound locating device is transparent.

17-24. (canceled)

25. A tracheostomy dressing as further defined in claim 2, wherein the tracheostomy dressing is impregnated with one or more antiseptic chemicals.

26. A tracheostomy dressing as defined in claim 1, wherein the textured portion has a surface feel that is distinct from the surrounding area

27. A tracheostomy dressing as defined in claim 13, wherein the textured portion is circular in shape, recessed and ribbed.
